(19) United States
(12) Patent Application Publication
(10) Pub. No.: US 2010/0121404 A1
(43) Pub. Date: May 13, 2010

(54) IMPLANTABLE HEART STIMULATING DEVICE

(75) Inventors: Anders Björling, Solna (SE); Karin Järverud, Solna (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St Jude Medical AB

(21) Appl. No.: 12/595,919

(22) PCT Filed: Apr. 24, 2007
(86) PCT No.: PCT/SE2007/000398

§ 371 Date: Oct. 14, 2009

(51) Int. Cl. A61N 1/37

(52) U.S. Cl. 607/28

(57) ABSTRACT

Implantable heart stimulating device has at least one electrode lead provided with at least two electrodes adapted to be arranged for electrical stimulation of a heart, a pulse generating that applies stimulation pulses between the electrodes, wherein one of the electrodes is the cathode and the other is the anode, to achieve cathodal capture of heart tissue by the cathode electrode. An anodal capture detector detects anodal capture at the anode electrode. The device further has a control unit and if anodal capture is detected by the detection means, the control unit changes the pacing regimen in order to optimize the hemodynamics of the heart.

### BACKGROUND OF THE INVENTION

1. Field of the Invention

The present invention relates to an implantable heart stimulating device and a method suitable for detecting anodal capture and for changing the pacing regimen of the device in order to optimize the hemodynamics of the heart.

2. Description of the Prior Art

When stimulating LV-tip to RV-ring in a biventricular system a so called anodal stimulation generating an anodal capture may occur on the RV-ring. If the left ventricle is stimulated first—which it often is—both ventricles will depolarize at the same time and a ventricle-ventricle (VV) delay optimization is then not possible to perform.

Furthermore, an automatic capture algorithm may detect loss of capture at each RV stimulation since the RV has already been stimulated and is thus refractory. This, in turn, will lead to unnecessary going into high output mode and incorrect diagnostics.

As will be discussed in detail in the following the above is related to that the unipolar voltage strength-duration curves for the LV tip and the RV ring electrodes have different shapes. Anodal thresholds are normally higher than cathodal thresholds for the same electrode. The LV thresholds are normally higher than RV thresholds and the ring thresholds are normally higher than the tip thresholds because of different surface area and distance to excitable tissue.

All these circumstances influence the bifocal stimulation thresholds so that anodal threshold may be higher than cathodal at short pulse width, while the cathodal threshold may be higher for a long pulse width.

In order to fully explain the present invention a general background will be given in the following.

In order to excite the left ventricle, the lead must be disposed near the left ventricle, preferably in the region of the free lateral or posterior wall, which may most easily be accomplished by placing the lead through the coronary sinus and into a left cardiac vein. Unlike a lead for the right ventricle, which is disposed within the ventricle where a tip electrode can be fixed into the myocardium, the electrodes of a lead in a cardiac vein cannot be fixed into the myocardium since that would require puncturing the vein. Instead, in the case of a bipolar lead, both the tip and ring electrodes (or proximal and distal electrodes in the case where both electrodes are ring electrodes or other types of structures) are positioned within the vein adjacent to the left ventricular myocardium. Because it is fixed into the myocardium, the tip of a bipolar lead in the right ventricle has a lower capture threshold than the ring electrode. Normally, therefore, the tip of a bipolar lead is used as the cathode in order to achieve the desirable cathodal capture when a voltage pulse is impressed across the two electrodes. With a bipolar lead in a cardiac vein, on the other hand, both electrodes are external to the myocardium and may have similar capture thresholds so that either anodal or cathodal capture can occur when a pacing pulse is output through the lead. Cathodal capture means that cathodal stimulation is responsible for the contraction. It has been found, however, that anodal thresholds increase over time so that eventually only the desired cathodal capture will occur. Nevertheless, a problem arises when the pulse energy for a bipolar lead in a cardiac vein is adjusted. When the lead is implanted, the capture threshold for the tip or distal electrode (i.e., the electrode usually selected to function as the cathode) may be higher than that of the ring or proximal electrode. When the clinician then determines the capture threshold of the lead with a bipolar pulse in order to adjust the stimulus pulse energy, it is impossible to distinguish between anodal and cathodal capture. There is then a risk that the stimulus pulse energy will be set to an anodal capture threshold when the cathodal capture threshold is higher. As the anodal capture threshold increases over time, the stimulation pulses may no longer be of sufficient energy to excite the left ventricle (diminishing or eliminating the programmed safety margin), and the patient may experience sporadic or total loss of resynchronization therapy.

U.S. Pat. No. 6,687,545 discloses a cardiac stimulation system and method for performing automatic capture verification during bipolar stimulation by eliminating capture verification during a cardiac cycle in which anodal stimulation is detected. Anodal stimulation is detected by the absence of a delay between the bipolar stimulation pulse and an evoked response sensed at the electrode functioning as the anode during stimulation. Automatic capture verification during bipolar stimulation is recommended only if anodal stimulation is not detected at a working stimulation output. During automatic capture verification, if anodal stimulation is detected, a capture threshold search is performed.

In the method and device described in U.S. Pat. No. 6,687,545, unipolar sensing is performed using e.g. the right ventricular ring electrode and the housing to determine if a stimulation pulse produced anodal stimulation at the ring electrode.

According to this patent, this is performed by determining the time from the stimulation pulse to the onset of the evoked response. Typically, a 20 to 40 ms conduction delay to the unipolar ring evoked response signal occurs when only cathodal stimulation is present. Therefore, if there is a delay to the evoked response as determined then anodal stimulation is not indicated and will not interfere with evoked response detection during bipolar evoked response sensing of the bipolar stimulation at the currently programmed output. If no delay to the evoked response is measured then anodal stimulation is occurring at the ring electrode at the programmed stimulation output. Thus, the system and method disclosed in U.S. Pat. No. 6,687,545 may be used to detect and to verify anodal stimulation.

U.S. Pat. No. 6,611,712 discloses an apparatus and method for testing the capture threshold of a bipolar lead of a cardiac rhythm management device in order to determine an appropriate stimulus pulse energy for the lead and/or select an appropriate stimulation configuration.

### SUMMARY OF THE INVENTION

The present invention follows drawbacks that have been identified as to how presently used devices react upon detected anodal capture and consequently an object of the present invention is primarily to eliminate the adverse hemodynamic consequences of anodal capture.

The above object is achieved in accordance with the present invention by an implantable heart stimulating device and method wherein at least one electrode lead, carrying at least two electrodes, is implanted in vivo and is connected to a pulse generator that applies stimulation pulses between the electrodes to electrically stimulate the heart of a patient. The electrodes have a polarity that defines one of the electrodes as the cathode and the other as the anode, and the pulse generator is operated to achieve cathodal capture of heart tissue by the cathode electrode. A capture detection procedure is implemented to detect anodal capture at the anode electrode. A control unit, that operates the pulse generator, is supplied with an indication as to if and when anodal capture occurs, and if anodal capture has occurred, the control unit changes the pacing regimen that is used to operate the pulse generator in order to optimize the hemodynamics of the heart.

The present invention achieved solutions where automatic electronic repositioning of stimulation site is performed in order to enhance hemodynamic performance, e.g. when cardiac remodelling occurs during CRT. The solution according to the invention is to use a bipolar lead for stimulating the left ventricle and with the use of minimum stimulation energy (e.g. acquired through threshold search methods).

In the situations where anodal stimulation on the LV ring occurs, this is important information which may affect the device therapy and pacing settings. As an example, the pacing output can be decreased by switching polarity since the cathodal threshold is normally lower than the anodal threshold.

Furthermore, since the AV and VV delays are based on a certain activation sequence, they need to be changed for optimal pacemaker therapy if the LV stimulation site is changed from LV tip to LV ring.

The occurrence of anodal capture could be due to a change in the patient's health status—e.g. previously dormant tissue may have become viable—which may result in that anodal capture may be taken as an indication to e.g. further investigate the patient's heart failure (HF) status.

As mentioned above, the focus of the present invention is on the hemodynamic effects and health status assessment, although saving energy in the implanted device is another important benefit.

Everything described herein would also be applicable to bipolar RV leads or RA leads as well. However, the probability of anodal stimulation occurring in these leads is normally very small. Therefore, the invention is primarily described by the example of using LV leads, but is equally applicable to other types of leads.

### BRIEF DESCRIPTION OF THE DRAWINGS

FIG. 1 shows a block diagram schematically illustrating the present invention.

FIG. 2 is a block diagram illustrating a preferred embodiment of the present invention.

### DESCRIPTION OF THE PREFERRED EMBODIMENTS

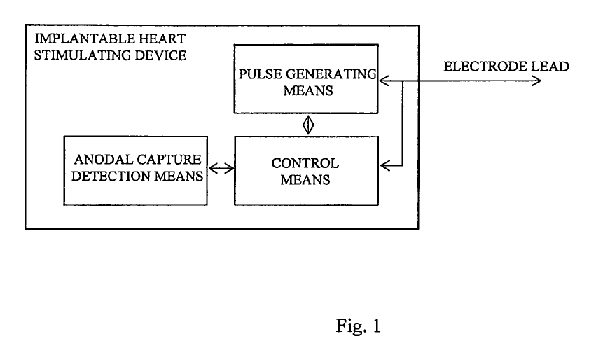

With references to FIG. 1 the present invention relates to an implantable heart stimulating device having at least one electrode lead provided with at least two electrodes (not shown) adapted to be arranged for electrical stimulation of a heart, and also for sensing electrical potentials of heart tissue. Preferably, these electrodes are adapted for stimulation/sensing of the left ventricle (LV) of the heart.

The device further has a pulse generator that applies stimulation pulses between the electrodes, where one of the electrodes being the cathode and the other being the anode, to achieve cathodal capture of heart tissue by the cathode electrode, and an anodal capture detector that detects anodal capture at the anode electrode.

The device further has a control unit. If anodal capture is detected by the anodal capture detector, the control unit changes the pacing regimen in order to optimize the hemodynamics of the heart.

According to one embodiment of the present invention the pacing regime is changed by changing predefined pacing parameters being specified pacing intervals, e.g. the AV and/or the VV interval.

According to another embodiment the pacing regime is changed by switching polarities of the electrodes, i.e. such that the anode electrode being the cathode electrode and vice versa.

Naturally, these embodiments may also be combined such that the stimulation regime is changed by switching polarities of said electrodes and then by changing predefined pacing parameters, e.g. specified pacing intervals, in relation thereto.

Often, and in addition to changing the pacing regimen, a stimulation threshold search has to be performed. The person skilled in the art is familiar to numerous different types of threshold search methods that would be applicable in that respect.

Detection of anodal stimulation on e.g. the LV ring can be made either at follow-ups when the physician requests such an investigation of the capture status, or continuously during the device's operation.

According to one embodiment the anodal capture detector performs a so-called paced depolarization integral (PDI) initialization test in order detect anodal capture.

Studies have shown that the paced depolarization integral (PDI) is a parameter which provides important information about certain cardiac functions. This parameter is obtained, by applying a pacing pulse to the ventricle and integrating a portion of the evoked cardiac electrogram. It has been experimentally determined that the PDI parameter is proportional to a number of physiological characteristics. (See Steinhaus and Nappholz, THE INFORMATION CONTENT OF THE CARDIAC ELECTROGRAM AT THE STIMULUS SITE, Proceedings of the Twelfth Annual International Conference of the I.E.E.E. Engineering Medicine and Biology Society, Vol. 12, No. 2, 1990, pp. 0607-0609.)

It has been seen that during a so-called PDI initialization test which is performed in order to assess the ability to enable (biventricular) capture, anodal stimulation at the LV ring can be identified. During the PDI initialization procedure, the PDI values (areas) for different pacing amplitudes are collected and analyzed. At a pulse width of 0.5 ms, amplitudes from 0 to 4.5 V are typically investigated. In case of cathodal stimulation only, the PDI vs. amplitude plot shows two distinct plateaus—one at low amplitudes (loss of capture) and one at high amplitudes ((cathodal) capture).

In case of anodal stimulation, however, three plateaus may occur—one from loss, one from cathodal or anodal capture, and one from cathodal and anodal capture. During “double” capture, the ER morphology is different from when cathodal or anodal capture occurs and the area (PDI) is typically larger.

Not only the PDI method, but other methods as well, based on morphological features of the ER may be able to use in order to detect anodal stimulation.

In accordance with another embodiment the anodal capture detection means is adapted to measure the temporal distance between an applied stimulation pulse and a predetermined portion of the evoked response signal and anodal capture is considered detected if a predefined temporal criterion is fulfilled. This is based upon the fact that the distance from the pacing pulse to the minimum value of the ER in the unipolar signal (LV-tip to case) is longer during anodal capture than during cathodal capture. The opposite is true, i.e. the temporal distance is shorter, for the IEGM measured between the LV-ring and the case. This phenomenon may be utilized for continuous detection of anodal stimulation. See e.g. the above-referenced U.S. Pat. No. 6,687,545.

As an example, if the ER morphology in the LV ring to case signal changes, particularly if the interval from stimulation to ER minimum decreases, it is likely that anodal capture has occurred. This is an example of how detection of anodal stimulation can be detected during operation of the device. Several other morphologically based detection methods may be used.

To further increase the safety of the device, the anodal capture detector includes, according to an alternative embodiment, an anodal capture confirmation unit (not shown in FIG. 1) that confirms detected anodal capture at the anode electrode. The anodal confirmation is performed by the anodal capture confirmation unit by, for one stimulation pulse, using another electrode as cathode electrode, e.g. the indifferent electrode at the housing, and by keeping the anodal electrode, and if anodal capture still occurs anodal capture is confirmed. The stimulation configuration may e.g. be changed for one stimulation pulse to LV-ring to housing electrode (LV-ring positive and housing electrode negative). If capture not is acquired for this configuration, a backup pulse may be supplied between the LV-tip and LV-ring or any other configuration to assure ventricular contraction.

However, if capture is acquired with the investigated stimulation configuration, then anodal capture is confirmed.

Regardless of which anodal capture detection test is performed, the anodal capture detector automatically performs such a test continuously, at regular intervals or when specified situations occur, e.g. at follow-up procedures. If an anodal capture is detected as a result of the test, an anodal capture alert flag is set to indicate at a follow-up procedure that anodal capture has been detected. In that case, relevant parts of a detected electrocardiogram may be stored in a storing means, included in the control means, if an anodal capture alert flag is set. The stored electrocardiogram may preferably be transferred to an external programming device to be further evaluated.

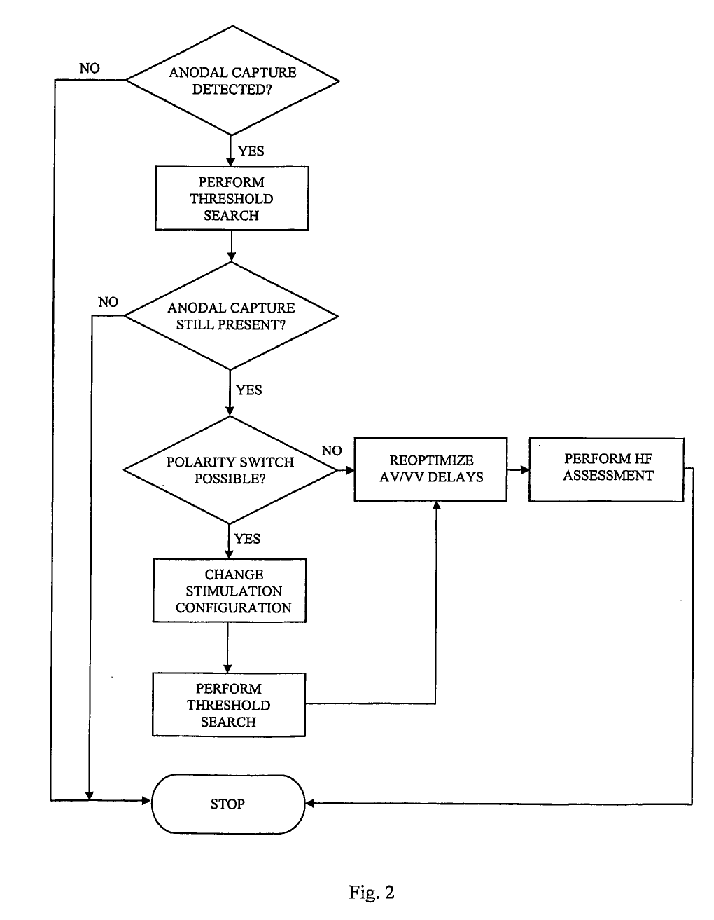

FIG. 2 shows a flow diagram of one embodiment of the present invention. In particular the figure illustrates a method in an implantable heart stimulating device having at least one electrode lead provided with at least two electrodes adapted to be arranged for electrical stimulation of a heart. This is achieved by use of a pulse generator that applies stimulation pulses between the electrodes, where one of the electrodes being the cathode and the other being the anode, to achieve cathodal capture of heart tissue by the cathode electrode.

The implantable heart stimulating device is provided with an anodal capture detector to detect anodal capture at the anode electrode

Upon detection of anodal capture an optional threshold search is performed and if still anodal capture is detected, the pacing regimen is changed, by the control means, in order to optimize the hemodynamics of the heart.

Preferably, it is evaluated if a polarity switch is possible. If so, the stimulation configuration is changed, e.g. the anode will become the cathode, and vice versa, and a threshold search is performed the new configuration.

In addition the pacing regimen is changed by re-optimizing specified pacing intervals, e.g. the AV and/or the VV interval. As indicated in the flow diagram of FIG. 2, the pacing regimen may be changed by switching polarities of the electrodes and then by changing predefined pacing parameters, e.g. specified pacing intervals, in relation thereto.

As also indicated in the flow diagram and discussed above, in addition to changing pacing regimen, irrespectively which type of change is performed, a stimulation threshold search is preferably performed using the new pacing regimen.

In order to detect anodal capture the anodal capture detector, according to one embodiment, performs a paced depolarization integral (PDI) initialization test (as discussed above).

According to another embodiment the anodal capture detector measures the temporal distance between an applied stimulation pulse and a predetermined portion of the evoked response signal and anodal capture is considered detected if a predefined temporal criterion is fulfilled, this detection method is also discussed above.

In the flow diagram is also illustrated the possibility to perform a heart failure (HF) assessment when relevant changes of the pacing regimen has been performed. The degree of HF may automatically be assessed by the implanted device. This may be done using different methods. One of these methods is to study the patient's activity trend. By studying the output of the activity sensor of the implantable stimulator (normally used for rate responsive control) a measure of the patient's physical activity level can be acquired. This has been shown to correlate well with the degree of heart failure. Another HF assessment tool can be to study the heart rate variability (HRV). The HRV is the variability of the patient's heart rate, which is under direct neural as well as hormonal control. The HRV has been shown to decrease with increasing levels of HF. A third method to assess the degree of heart failure is using impedance based upon intrathoracic impedance measurements for the early detection of pulmonary edema. This too, as well as other impedance-based methods may be used to assess the HF degree.

Other methods also exist and these three examples are intended to serve only as that, i.e. examples.

In the following one illustrative example of an implementation in accordance with the present invention is given.

Initially, a threshold search is performed on the cathode electrode. If anodal capture suddenly has appeared, this could be due to changed cellular properties. In that case, it may be reasonable to believe that the stimulation threshold at the cathode has also changed. If this is the case, the stimulation amplitude may be lowered and still achieve capture, resulting in decreased battery drain and increased longevity. Sudden anodal stimulation could thus serve as a trigger of a threshold search in devices with threshold determining capabilities.

If anodal capture disappears after this, then no further actions are performed.

However, if anodal capture still sustains after the cathodal threshold search has been performed, the polarity could be switched for additional energy savings.

In some cases it may not be possible to change polarity, e.g. due to hardware issues or the fact that the anode is in fact some kind of sensor that does not allow pacing. If that is the case, then hemodynamic settings in the device, e.g. AV and VV delay, may still be re-optimized. It is highly likely that the existing settings were optimized at the time of implantation, or at the last follow-up when only cathodal capture was present. Since both anodal and cathodal capture is now present, the depolarization sequence is somewhat changed. This, and the fact that the anodal capture could have arisen due to changed cellular properties, indicates that a re-optimization of the settings may be performed.

Furthermore, the presence of anodal capture could indicate that the patient's functional status could have changed. Thus, an additional measurement of the patient's health status (e.g. heart failure) should be obtained.

If the polarity is switched, then a new threshold search has to be performed. This time, the anode will become the cathode and vice versa. Once this is done, a re-optimization of the device settings is made and a health index measurement is obtained.

All responses discussed above may be done either automatically by the device, or the device may alert the physician at the next follow-up to perform all of the steps. It is also possible to transmit the information that anodal capture has been detected using long-range telemetry or systems like Housecall.

Although modifications and changes may be suggested by those skilled in the art, it is the intention of the inventors to embody within the patent warranted heron all changes and modifications as reasonably and properly come within the scope of their contribution to the art.

We claim as our invention:

1-28. (canceled)

29. An implantable heart stimulating device comprising:
an electrode lead that carries two electrodes, said electrode lead being adapted for implantation in a patient with said electrodes in contact with heart tissue of the patient;
a pulse generator mechanically connected to said electrode lead and electrically connected to said electrodes to define a polarity of said electrodes with one of said electrodes being a cathode electrode and the other of said electrodes being an anode electrode, said pulse generator generating electrical pulses between said electrodes for in vivo electrical stimulation of the heart tissue;
a control unit that operates said pulse generator according to a pacing regimen designed to achieve cathodal capture of said heart tissue by said cathode electrode;
an anodal capture detection unit that detects anodal capture of said heart tissue at said anode electrode; and
said control unit being supplied with a signal from said anodal capture detection unit indicating if and when anodal capture occurs and said control unit, in response to said signal, automatically modifying said pacing regimen to optimize hemodynamics of the heart of the patient.

30. An implantable heart stimulating device as claimed in claim 29 wherein said pacing regimen embodies pacing parameters selected from the group consisting of an AV interval and VV interval, and wherein said control unit is configured to change said pacing regimen by changing at least one of said pacing parameters.

31. An implantable heart stimulating device as claimed in claim 29 wherein said control unit is configured to change said pacing regimen by changing said polarity of said electrodes.

32. An implantable heart stimulating device as claimed in claim 29 wherein said pacing regimen comprising pacing parameters selected from the group consisting of an AV interval and a VV interval, and wherein said control unit is configured to change said pacing regimen by first switching the polarity of said electrodes and, if anodal capture still occurs, changing at least one of said pacing parameters.

33. An implantable heart stimulating device as claimed in claim 29 wherein said control unit, in addition to changing said pacing regimen, is configured to perform a stimulation threshold search upon receipt of said signal from said anodal capture detection unit.

34. An implantable heart stimulating device as claimed in claim 29 wherein said anodal capture detection unit is configured to execute a paced depolarization integral initialization test to detect anodal capture.

35. An implantable heart stimulating device as claimed in claim 29 wherein said anodal capture detection unit is configured to measure a temporal duration between an applied stimulation pulse from said pulse generator and a predetermined portion of an evoked response signal, and to generate said signal indicating occurrence of anodal capture if said temporal duration satisfies a predetermined criterion.

36. An implantable heart stimulating device as claimed in claim 29 wherein said anodal capture detection unit comprises an anodal capture confirmation unit that confirms the occurrence of anodal capture at said anode electrode.

37. An implantable heart stimulating device as claimed in claim 36 wherein said anodal capture confirmation unit is configured to confirm anodal capture by, for one stimulation pulse, using another electrode as the cathode electrode instead of said one of said electrodes while maintaining said other of said electrodes as said anodal electrode, and to confirm anodal capture if anodal capture still occurs at said anodal electrode.

38. An implantable heart stimulating device as claimed in claim 36 comprising an implantable housing comprising an indifferent electrode, and wherein said anodal capture confirmation unit uses said indifferent electrode as said cathode electrode to confirm anodal capture.

39. An implantable heart stimulating device as claimed in claim 29 wherein said anodal capture detection unit is configured to automatically perform an anodal capture detection test at times selected from the group consisting of continuously, at regular intervals, or upon an occurrence of a predetermined cardiac situation.

40. An implantable heart stimulating device as claimed in claim 29 wherein said control unit sets an anodal capture alert flag upon receiving said signal from said anodal capture detection unit.

41. An implantable heart stimulating device as claimed in claim 40 comprising a memory, and wherein said control unit causes a predetermined portion of a detected electrocardiogram of the patient to be stored in said memory when said anodal capture alert flag is set.

42. An implantable heart stimulating device as claimed in claim 41 comprising a communication device configured to communicate with an external programming device to communicate the electrocardiogram stored in the memory to said external programming device.

43. An implantable heart stimulating device as claimed in claim 29 wherein said electrode lead is configured for implantation in the left ventricle of the heart of the patient.

44. A method for stimulating a heart comprising the steps of:
implanting an electrode lead that carries two electrodes, with said electrodes in contact with heart tissue of the patient;
mechanically and electrically connecting a pulse generator to said electrode lead and to said electrodes to define a polarity of said electrodes with one of said electrodes being a cathode electrode and the other of said electrodes being an anode electrode, and with said pulse generator, generating electrical pulses between said electrodes for in vivo electrical stimulation of the heart tissue;
automatically operating said pulse generator according to a pacing regimen designed to achieve cathodal capture of said heart tissue by said cathode electrode;
detecting anodal capture of said heart tissue at said anode electrode; and
from a control unit supplied with a signal indicating if and when anodal capture occurs and said control unit, automatically modifying said pacing regimen in response to said signal to optimize hemodynamics of the heart of the patient.

45. A method as claimed in claim 44 comprising, in said pacing regimen, using pacing parameters selected from the group consisting of an AV interval and VV interval and from said control unit, changing said pacing regimen by changing at least one of said pacing parameters.

46. A method as claimed in claim 44 comprising said control unit, changing said pacing regimen by changing said polarity of said electrodes.

47. A method as claimed in claim 44 comprising, in said pacing regimen, using pacing parameters selected from the group consisting of an AV interval and a VV interval and from said control unit, changing said pacing regimen by first switching the polarity of said electrodes and, if anodal capture still occurs, changing at least one of said pacing parameters.

48. A method as claimed in claim 44 comprising, from said control unit, in addition to changing said pacing regimen, performing a stimulation threshold search upon receipt of said signal.

49. A method as claimed in claim 44 comprising detecting anodal by executing a paced depolarization integral initialization test.

50. A method as claimed in claim 44 comprising detecting anodal capture by measuring a temporal duration between an applied stimulation pulse from said pulse generator and a predetermined portion of an evoked response signal, and generating said signal indicating occurrence of anodal capture if said temporal duration satisfies a predetermined criterion.

51. A method as claimed in claim 44 comprising automatically confirming anodal capture after a detected occurrence of anodal capture at said anode electrode.

52. A method as claimed in claim 51 comprising confirming anodal capture by, for one stimulation pulse, using another electrode as the cathode electrode instead of said one of said electrodes while maintaining said other of said electrodes as said anodal electrode, and confirming anodal capture if anodal capture still occurs at said anodal electrode.

53. A method as claimed in claim 52 comprising providing an implantable housing having an indifferent electrode, and using said indifferent electrode as said cathode electrode to confirm anodal capture.

54. A method as claimed in claim 44 comprising detecting anodal capture by automatically performing an anodal capture detection test at times selected from the group consisting of continuously, at regular intervals, or upon an occurrence of a predetermined cardiac situation.

55. A method as claimed in claim 44 comprising, in said control unit, automatically setting an anodal capture alert flag upon receiving said signal.

56. A method as claimed in claim 55 comprising, from said control unit causing a predetermined portion of a detected electrocardiogram of the patient to be stored in a memory when said anodal capture alert flag is set.

57. A method as claimed in claim 56 comprising establishing communicating between said control unit and an external programming device, and communicating the electrocardiogram stored in the memory to said external programming device.

58. A method as claimed in claim 44 comprising implanting said electrode lead in the left ventricle of the heart of the patient.
